(19) United States
(12) Patent Application Publication
(10) Pub. No.: US 2016/0175302 A1
(43) Pub. Date: Jun. 23, 2016

(54) MASITINIB FOR TREATING GASTRIC CANCER

(71) Applicant: AB SCIENCE, Paris (FR)

(72) Inventors: Alain MOUSSY, Paris (FR); Jean-Pierre KINET, Massachusetts, MA (US); Colin MANSFIELD, Ecully (FR)

(21) Appl. No.: 14/973,075

(22) Filed: Dec. 17, 2015

(30) Foreign Application Priority Data
Dec. 17, 2014 (EP) 14198673.7

(51) Int. Cl. A61K 31/496; A61K 31/513; A61K 31/475; A61K 9/00; A61K 31/7048; A61K 31/4745; A61K 31/519

(52) U.S. Cl. CPC A61K 31/496; A61K 31/4745; A61K 31/513; A61K 31/519; A61K 9/0053; A61K 31/7048; A61K 31/475

(57) ABSTRACT

The present invention relates to a method for treating gastric cancer in a subject in need thereof, comprising administering to the subject a therapeutically effective amount of a tyrosine kinase inhibitor, in combination with a therapeutically effective amount of a chemotherapeutic agent.

### FIELD OF INVENTION

The present invention relates to the treatment of gastric cancer. In particular the present invention relates to the treatment of gastric cancer using masitinib.

### BACKGROUND OF INVENTION

Upper gastrointestinal (GI) tract cancers originating in the esophagus, gastroesophageal (GE) junction, and stomach, constitute a major health problem around the world. In Japan, gastric cancer remains the most common type of cancer among men. However, the incidence has been declining globally and gastric cancer is one of the least common cancers in North America. Gastric cancer is often diagnosed at an advanced stage, because screening is not performed in most of the world, except in Japan where early detection is often done. Risk factors for gastric cancer are Helicobacter Pylori infection, smoking, high salt intake, and other dietary factors. A subset of gastric cancers (1% to 3%) is inherited (hereditary diffuse gastric cancer) with cadherin mutations occurring in approximately 25% of families with autosomal dominant predisposition to diffuse type gastric cancer.

When possible, surgery is the primary treatment option for gastric cancer. For more advanced tumor, perioperative chemotherapy with ECF (epirubicin, cisplatin, 5-fluorouracil) regimen is recommended as an added option to the standard of care. However, advanced or metastatic gastric carcinomas are nowadays incurable, and palliative interventions with chemotherapy can provide symptomatic relief and may result in significant improvement in nutritional status and overall quality of life. Single agents which are active in patients with advanced gastric cancer include 5-FU, mitomycin, etoposide, and cisplatin. Several newer agents and their combinations including paclitaxel, docetaxel, irinotecan, epirubicin, oxaliplatin, oral etoposide and UFT (combination of uracil and tegafur/prodrug of 5-FU) have shown activity against gastric cancer. Oral targeted agents also hold promise in the treatment of gastric cancer and are currently tested in ongoing clinical trials (bevacizumab and sorafenib). However these new targeted therapies have raised safety concerns and the benefits in terms of overall survival need to be confirmed. Indeed, most of these agents have been recently associated with toxicity to the heart and bowel perforation, hypertension and thromboembolic phenomenon is observed in patients treated with bevacizumab.

Therefore, there are still great needs for new efficient agents or for new combination strategies using safer and more efficient targeted agents to improve gastric cancer treatment and to circumvent resistance to chemotherapeutic agents.

Masitinib mesilate is a novel tyrosine kinase inhibitor part of 2-aminoarylthiazole derivatives that mainly targets c-Kit, and the angiogenic PDGF receptors but was also found to target the non-receptor tyrosine kinases Lyn and to a lower extent FGFR3 (Dubreuil et al. 2009).

The Applicant herein surprisingly demonstrates that masitinib potentiates the cytotoxic effect of chemotherapies in gastric cancer, including 5-fluorouracil (5-FU), irinotecan, etoposide, and vincristine. The present invention thus relates to the synergistic combination of masitinib and chemotherapeutic agents for treating gastric cancer.

### SUMMARY OF THE INVENTION

The present invention thus relates to a method for treating gastric cancer in a subject in need thereof, comprising administering to the subject a therapeutically effective amount of a tyrosine kinase inhibitor or mast cell inhibitor or a pharmaceutically acceptable salt or solvate thereof, optionally in combination with a therapeutically effective amount of at least one chemotherapeutic agent.

In one embodiment, the tyrosine kinase inhibitor or mast cell inhibitor is an inhibitor of c-Kit, Lyn, Fyn and/or PDGFR α and β. In one embodiment, the tyrosine kinase inhibitor or mast cell inhibitor is masitinib or a pharmaceutically acceptable salt or solvate thereof. In one embodiment, the tyrosine kinase inhibitor or mast cell inhibitor is masitinib mesilate.

In one embodiment, the method of the invention is for improving survival and/or life expectancy of the subject.

In one embodiment, the method of the invention comprises sensitizing to a chemotherapeutic agent or restoring sensitivity to a chemotherapy in the subject.

In one embodiment, gastric cancer is locally advanced gastric cancer or metastatic gastric cancer. In one embodiment, gastric cancer is primary gastric cancer, preferably gastric adenocarcinoma or gastro-esophageal junction adenocarcinoma. In one embodiment, gastric cancer is unresectable gastric adenocarcinoma or unresectable gastro-esophageal junction adenocarcinoma.

In one embodiment, gastric cancer is relapsed or is refractory gastric cancer.

In one embodiment, the subject is naïve to anti-gastric cancer treatments, or gastric cancer has relapsed after at least one anti-gastric cancer treatment, or after two or more anti-gastric cancer treatments.

In one embodiment, anti-gastric cancer treatment includes treatment with one or more chemotherapeutic agents, preferably selected from adrucil (fluorouracil), Cyramza (Ramucirumab), Docetaxel, Doxorubicin Hydrochloride, Efudex (Fluorouracil), Fluoroplex (Fluorouracil), Fluorouracil, Herceptin (Trastuzumab), Mitomycin C, Mitozytrex (Mitomycin C), Mutamycin (Mitomycin C), Ramucirumab, Taxotere (Docetaxel), Trastuzumab, FU-LV combination (combination of Fluorouracil and Leucovorin Calcium), TPF combination (combination of Docetaxel (Taxotere), Cisplatin (Platinol) and Fluorouracil), vincristine, folinic acid (leucovorin), epirubicin, cisplatin, 5-fluorouracil (5-FU), etoposide, paclitaxel, irinotecan, oxaliplatin, bevacizumab and sorefanib and mixtures thereof, or specific combinations of chemotherapeutics including the combination of 5-FU, folinic acid and irinotecan (FOLFIRI protocol), FU-LV combination (combination of Fluorouracil and Leucovorin Calcium), TPF combination (combination of Docetaxel (Taxotere), Cisplatin (Platinol) and Fluorouracil).

In one embodiment, the at least one chemotherapeutic agent is selected from 5-fluorouracil, irinotecan, folinic acid, etoposide, vincristine and mixtures thereof, such as, for example, the combination of 5-fluorouracil, irinotecan, folinic acid (FOLFIRI protocol).

In one embodiment, the therapeutically effective amount of the tyrosine kinase inhibitor is about 6 mg/kg/day.

In one embodiment, the tyrosine kinase inhibitor is orally administered.

In one embodiment, the tyrosine kinase inhibitor is administered twice daily.

The present invention also relates to a method for inhibiting tyrosine kinases, preferably selected from the group consisting of c-Kit, LYN, FYN and PDGFR α and β and for inducing an anti-tumoral Th1 immune response, in a gastric cancer patient, thereby treating gastric cancer, wherein said method comprises administering a therapeutically effective amount of a tyrosine kinase inhibitor or mast cell inhibitor, preferably masitinib, or a pharmaceutically acceptable salt or solvate thereof, optionally in combination with a therapeutically effective amount of a chemotherapeutic agent.

The present invention also relates to a pharmaceutical composition comprising a tyrosine kinase inhibitor or mast cell inhibitor or a pharmaceutically acceptable salt or solvate thereof and a chemotherapeutic agent, in combination with at least one pharmaceutically acceptable carrier, wherein said tyrosine kinase inhibitor or mast cell inhibitor is preferably masitinib mesilate, and wherein said chemotherapeutic agent is preferably selected from doxorubicin, 5-fluorouracil, irinotecan, folinic acid, etoposide, vincristine and mixtures thereof.

The present invention also relates to a medicament comprising a tyrosine kinase inhibitor or mast cell inhibitor or a pharmaceutically acceptable salt or solvate thereof and a chemotherapeutic agent, wherein said tyrosine kinase inhibitor or mast cell inhibitor is preferably masitinib mesilate, and wherein said chemotherapeutic agent is preferably selected from doxorubicin, 5-fluorouracil, irinotecan, folinic acid, etoposide, vincristine and mixtures thereof.

The present invention also relates to a kit of part comprising, in a first part, a tyrosine kinase inhibitor or mast cell inhibitor or a pharmaceutically acceptable salt or solvate thereof, preferably wherein said tyrosine kinase inhibitor or mast cell inhibitor is masitinib mesilate, and, in a second part, a chemotherapeutic agent, preferably selected from doxorubicin, 5-fluorouracil, irinotecan, folinic acid, etoposide, vincristine and mixtures thereof.

The present invention also relates to the pharmaceutical composition as described hereinabove, the medicament as described hereinabove or the kit of part as described hereinabove, for treating gastric cancer.

### DEFINITIONS

In the present invention, the following terms have the following meanings:

The term “subject” refers to a mammal, preferably a human. In one embodiment, a subject may be a “patient”, i.e. a warm-blooded animal, more preferably a human, who/which is awaiting the receipt of, or is receiving medical care or was/is/will be the object of a medical procedure, or is monitored for the development of a gastric cancer. In one embodiment, the subject is an adult (for example a subject above the age of 18). In another embodiment, the subject is a child (for example a subject below the age of 18). In one embodiment, the subject is a male. In another embodiment, the subject is a female.

The term “treating” or “treatment” refers to both therapeutic treatment and prophylactic or preventative measures; wherein the object is to prevent or slow down (lessen) gastric cancer. Those in need of treatment include those already with gastric cancer as well as those prone to have gastric cancer or those in whom gastric cancer is to be prevented. A subject is successfully “treated” for gastric cancer if, after receiving a therapeutic amount of a tyrosine kinase inhibitor or mast cell inhibitor according to the methods of the present invention, the patient shows observable and/or measurable reduction in or absence of one or more of the following: reduction in the number of pathogenic cells; reduction in the percent of total cells that are pathogenic; and/or relief to some extent, of one or more of the symptoms associated with gastric cancer; reduced morbidity and mortality, and improvement in quality of life issues. The above parameters for assessing successful treatment and improvement in the disease are readily measurable by routine procedures familiar to a physician.

The term “therapeutically effective amount” means the level or amount of agent that is aimed at, without causing significant negative or adverse side effects to the target, (1) delaying or preventing the onset of gastric cancer; (2) slowing down or stopping the progression, aggravation, or deterioration of one or more symptoms of gastric cancer; (3) bringing about ameliorations of the symptoms of gastric cancer; (4) reducing the severity or incidence of gastric cancer; or (5) curing gastric cancer. A therapeutically effective amount may be administered prior to the onset of gastric cancer, for a prophylactic or preventive action. Alternatively or additionally, the therapeutically effective amount may be administered after initiation of gastric cancer, for a therapeutic action or maintenance of a therapeutic action.

The term “pharmaceutically acceptable carrier or excipient” refers to an excipient or carrier that does not produce an adverse, allergic or other untoward reaction when administered to an animal, preferably a human. It includes any and all solvents, dispersion media, coatings, antibacterial and antifungal agents, isotonic and absorption delaying agents and the like. For human administration, injected preparations should meet sterility, pyrogenicity, general safety and purity standards as required by regulatory offices, such as, for example, FDA Office or EMA.

The term “about” preceding a figure means plus or less 10% of the value of said figure.

As used herein, the term an “aryl group” means a monocyclic or polycyclic-aromatic radical comprising carbon and hydrogen atoms. Examples of suitable aryl groups include, but are not limited to, phenyl, tolyl, anthracenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents. In one embodiment, the aryl group is a monocyclic ring, wherein the ring comprises 6 carbon atoms, referred to herein as “(C6)aryl”.

As used herein, the term “alkyl group” means a saturated straight chain or branched non-cyclic hydrocarbon having from 1 to 10 carbon atoms. Representative saturated straight chain alkyls include methyl, ethyl, n-propyl, n-butyl, n-pentyl, n-hexyl, n-heptyl, n-octyl, n-nonyl and n-decyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, 2,3-dimethylpentyl, 2,4-dimethylpentyl, 2,3-dimethylhexyl, 2,4-dimethylhexyl, 2,5-dimethylhexyl, 2,2-dimethylpentyl, 2,2-dimethylhexyl, 3,3-dimtheylpentyl, 3,3-dimethylhexyl, 4,4-dimethylhexyl, 2-ethylpentyl, 3-ethylpentyl, 2-ethylhexyl, 3-ethylhexyl, 4-ethylhexyl, 2-methyl-2-ethylpentyl, 2-methyl-3-ethylpentyl, 2-methyl-4-ethylpentyl, 2-methyl-2-ethylhexyl, 2-methyl-3-ethylhexyl, 2-methyl-4-ethylhexyl, 2,2-diethylpentyl, 3,3-diethylhexyl, 2,2-diethylhexyl, 3,3-diethylhexyl and the like. Alkyl groups included in compounds of this invention may be optionally substituted with one or more substituents.

As used herein, the term “alkoxy” refers to an alkyl group which is attached to another moiety by an oxygen atom. Examples of alkoxy groups include methoxy, isopropoxy, ethoxy, tert-butoxy, and the like. Alkoxy groups may be optionally substituted with one or more substituents.

As used herein, the term “heteroaryl” or like terms means a monocyclic or polycyclic heteroaromatic ring comprising carbon atom ring members and one or more heteroatom ring members (such as, for example, oxygen, sulfur or nitrogen). Typically, a heteroaryl group has from 1 to about 5 heteroatom ring members and from 1 to about 14 carbon atom ring members. Representative heteroaryl groups include pyridyl, 1-oxo-pyridyl, furanyl, benzo[1,3]dioxolyl, benzo[1,4]dioxinyl, thienyl, pyrrolyl, oxazolyl, imidazolyl, thiazolyl, isoxazolyl, quinolinyl, pyrazolyl, isothiazolyl, pyridazinyl, pyrimidinyl, pyrazinyl, triazinyl, triazolyl, thiadiazolyl, isoquinolinyl, indazolyl, benzoxazolyl, benzofuryl, indolizinyl, imidazopyridyl, tetrazolyl, benzimidazolyl, benzothiazolyl, benzothiadiazolyl, benzoxadiazolyl, indolyl, tetrahydroindolyl, azaindolyl, imidazopyridyl, quinazolinyl, purinyl, pyrrolo[2,3]pyrimidinyl, pyrazolo[3,4]pyrimidinyl, imidazo[1,2-a]pyridyl, and benzo(b)thienyl. A heteroatom may be substituted with a protecting group known to those of ordinary skill in the art, for example, the hydrogen on a nitrogen may be substituted with a tert-butoxycarbonyl group. Heteroaryl groups may be optionally substituted with one or more substituents. In addition, nitrogen or sulfur heteroatom ring members may be oxidized. In one embodiment, the heteroaromatic ring is selected from 5-8 membered monocyclic heteroaryl rings. The point of attachment of a heteroaromatic or heteroaryl ring to another group may be at either a carbon atom or a heteroatom of the heteroaromatic or heteroaryl rings.

The term “heterocycle” as used herein, refers collectively to heterocycloalkyl groups and heteroaryl groups.

As used herein, the term “heterocycloalkyl” means a monocyclic or polycyclic group having at least one heteroatom selected from O, N or S, and which has 2-11 carbon atoms, which may be saturated or unsaturated, but is not aromatic. Examples of heterocycloalkyl groups include (but are not limited to): piperidinyl, piperazinyl, 2-oxopiperazinyl, 2-oxopiperidinyl, 2-oxopyrrolidinyl, 4-piperidonyl, pyrrolidinyl, hydantoinyl, valerolactamyl, oxiranyl, oxetanyl, tetrahydropyranyl, tetrahydrothiopyranyl, tetrahydropyrindinyl, tetrahydropyrimidinyl, tetrahydrothiopyranyl sulfone, tetrahydrothiopyranyl sulfoxide, morpholinyl, thiomorpholinyl, thiomorpholinyl sulfoxide, thiomorpholinyl sulfone, 1,3-dioxolane, tetrahydrofuranyl, dihydrofuranyl-2-one, tetrahydrothienyl, and tetrahydro-1,1-dioxothienyl. Typically, monocyclic heterocycloalkyl groups have 3 to 7 members. Preferred 3 to 7 membered monocyclic heterocycloalkyl groups are those having 5 or 6 ring atoms. A heteroatom may be substituted with a protecting group known to those of ordinary skill in the art, for example, the hydrogen on a nitrogen may be substituted with a tert-butoxycarbonyl group. Furthermore, heterocycloalkyl groups may be optionally substituted with one or more substituents. In addition, the point of attachment of a heterocyclic ring to another group may be at either a carbon atom or a heteroatom of a heterocyclic ring. Only stable isomers of such substituted heterocyclic groups are contemplated in this definition.

As used herein the term “substituent” or “substituted” means that a hydrogen radical on a compound or group is replaced with any desired group that is substantially stable to reaction conditions in an unprotected form or when protected using a protecting group. Examples of preferred substituents are those found in the exemplary compounds and embodiments disclosed herein, as well as halogen (chloro, iodo, bromo, or fluoro); alkyl; alkenyl; alkynyl; hydroxy; alkoxy; nitro; thiol; thioether; imine; cyano; amido; phosphonato; phosphine; carboxyl; thiocarbonyl; sulfonyl; sulfonamide; ketone; aldehyde; ester; oxygen (—O); haloalkyl (e.g., trifluoromethyl); cycloalkyl, which may be monocyclic or fused or non-fused polycyclic (e.g., cyclopropyl, cyclobutyl, cyclopentyl, or cyclohexyl), or a heterocycloalkyl, which may be monocyclic or fused or non-fused polycyclic (e.g., pyrrolidinyl, piperidinyl, piperazinyl, morpholinyl, or thiazinyl), monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidinyl, benzimidazolyl, benzothiophenyl, or benzofuranyl); amino (primary, secondary, or tertiary); CO₂CH₃; CONH₂; OCH₂CONH₂; NH₂; SO₂NH₂; OCHF₂; CF₃; OCF₃; and such moieties may also be optionally substituted by a fused-ring structure or bridge, for example —OCH₂O—. These substituents may optionally be further substituted with a substituent selected from such groups. In certain embodiments, the term “substituent” or the adjective “substituted” refers to a substituent selected from the group consisting of an alkyl, an alkenyl, an alkynyl, an cycloalkyl, an cycloalkenyl, a heterocycloalkyl, an aryl, a heteroaryl, an aralkyl, a heteraralkyl, a haloalkyl, —C(O)NR₁₁R₁₂, —NR₁₃C(O)R₁₄, a halo, —OR₁₃, cyano, nitro, a haloalkoxy, —C(O)R₁₃, —NR₁₁R₁₂, —SR₁₃, —C(O)OR₁₃, —OC(O)R₁₃, —NR₁₃C(O)NR₁₁R₁₂, —OC(O)NR₁₁R₁₂, —NR₁₃C(O)OR₁₄, —S(O)rR₁₃, —NR₁₃S(O)rR₁₄, —OS(O)rR₁₄, S(O)rNR₁₁R₁₂, —O, —S, and —N—R₁₃, wherein r is 1 or 2; R₁₁ and R₁₂, for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl; or R₁₁ and R₁₂ taken together with the nitrogen to which they are attached are optionally substituted heterocycloalkyl or optionally substituted heteroaryl; and R₁₃ and R₁₄ for each occurrence are, independently, H, an optionally substituted alkyl, an optionally substituted alkenyl, an optionally substituted alkynyl, an optionally substituted cycloalkyl, an optionally substituted cycloalkenyl, an optionally substituted heterocycloalkyl, an optionally substituted aryl, an optionally substituted heteroaryl, an optionally substituted aralkyl, or an optionally substituted heteraralkyl. In certain embodiments, the term “substituent” or the adjective “substituted” refers to a solubilising group.

The term “solubilising group” means any group which can be substantially ionized and that enables the compound to be soluble in a desired solvent, such as, for example, water or water-containing solvent. Furthermore, the solubilising group can be one that increases the compound or complex's lipophilicity. Typically, the solubilising group is selected from alkyl group substituted with one or more heteroatoms such as N, O, S, each optionally substituted with alkyl group substituted independently with alkoxy, amino, alkylamino, dialkylamino, carboxyl, cyano, or substituted with cycloheteroalkyl or heteroaryl, or a phosphate, or a sulfate, or a carboxylic acid. For example, by “solubilising group” it is referred herein to one of the following:
an alkyl, cycloalkyl, aryl, heretoaryl group comprising either at least one nitrogen or oxygen heteroatom or which group is substituted by at least one amino group or oxo group;
an amino group which may be a saturated cyclic amino group which may be substituted by a group consisting of alkyl, alkoxycarbonyl, halogen, haloalkyl, hydroxyalkyl, amino, monoalkylamino, dialkylamino, carbamoyl, monoalkylcarbamoyl and dialkylcarbamoyl;
one of the structures a) to i) shown below, wherein the wavy line and the arrow line correspond to the point of attachment to core structure of formula (A) or (B)

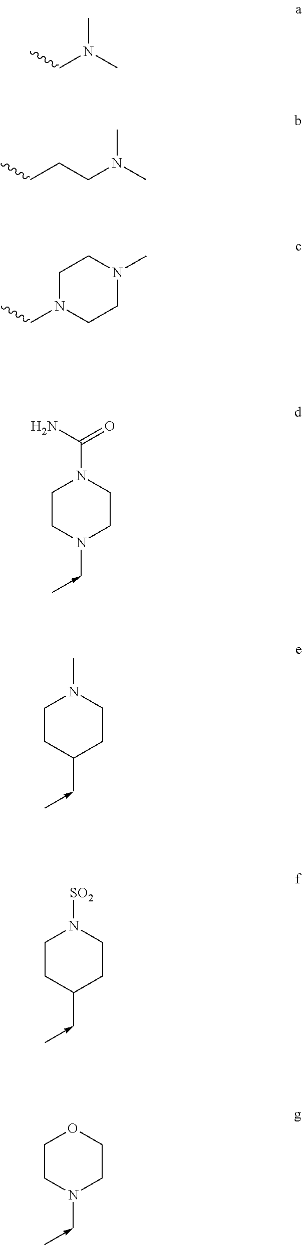

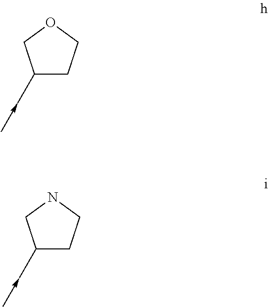

The term “cycloalkyl” means a saturated cyclic alkyl radical having from 3 to 10 carbon atoms. Representative cycloalkyls include cyclopropyl, 1-methylcyclopropyl, cyclobutyl, cyclopentyl, cyclohexyl, cycloheptyl, cyclooctyl, cyclononyl, and cyclodecyl. Cycloalkyl groups can be optionally substituted with one or more substituents.

The term “halogen” means —F, —Cl, —Br or —I.

### DETAILED DESCRIPTION

The present invention thus relates to a method for treating gastric cancer in a subject in need thereof, wherein said method comprises administering to the subject a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof.

In one embodiment, the tyrosine kinase inhibitor of the invention is a c-Kit inhibitor. The present invention thus also relates to a c-Kit inhibitor for treating gastric cancer.

Tyrosine kinases are receptor type or non-receptor type proteins, which transfer the terminal phosphate of ATP to tyrosine residues of proteins thereby activating or inactivating signal transduction pathways. These proteins are known to be involved in many cellular mechanisms, which in case of disruption, lead to disorders such as abnormal cell proliferation and migration as well as inflammation. A tyrosine kinase inhibitor is a drug that inhibits tyrosine kinases, thereby interfering with signaling processes within cells. Blocking such processes can stop the cell growing and dividing.

In one embodiment, the tyrosine kinase inhibitor of the invention has the following formula (A):

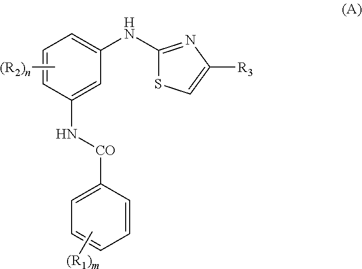

wherein
R₁ and R₂, are selected independently from hydrogen, halogen, a linear or branched alkyl, cycloalkyl group containing from 1 to 10 carbon atoms, trifluoromethyl, alkoxy, cyano, dialkylamino, and a solubilising group, m is 0-5 and n is 0-4;
the group R₃ is one of the following:
   i. an aryl group such as phenyl or a substituted variant thereof bearing any combination, at any one ring position, of one or more substituents such as halogen, alkyl groups containing from 1 to 10 carbon atoms, trifluoromethyl, cyano and alkoxy;
   ii. a heteroaryl group such as 2, 3, or 4-pyridyl group, which may additionally bear any combination of one or more substituents such as halogen, alkyl groups containing from 1 to 10 carbon atoms, trifluoromethyl and alkoxy;
   iii. a five-membered ring aromatic heterocyclic group such as for example 2-thienyl, 3-thienyl, 2-thiazolyl, 4-thiazolyl, 5-thiazolyl, which may additionally bear any combination of one or more substituents such as halogen, an alkyl group containing from 1 to 10 carbon atoms, trifluoromethyl, and alkoxy;

or a pharmaceutically acceptable salt or solvate thereof.

In one embodiment the tyrosine kinase inhibitor of the invention has general formula (B),

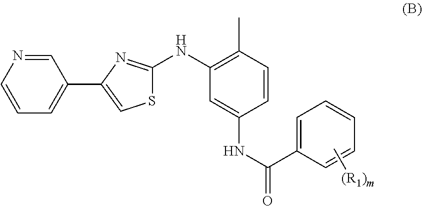

wherein:
R₁ is selected independently from hydrogen, halogen, a linear or branched alkyl, cycloalkyl group containing from 1 to 10 carbon atoms, trifluoromethyl, alkoxy, amino, alkylamino, dialkylamino, solubilising group;
m is 0-5;

or a pharmaceutically acceptable salt or solvate thereof.

In one embodiment, the tyrosine kinase inhibitor of formula (B) is masitinib or a pharmaceutically acceptable salt or solvate thereof, more preferably masitinib mesilate.

The present invention thus also relates to masitinib or a pharmaceutically acceptable salt or solvate thereof, more preferably masitinib mesilate for treating gastric cancer.

Pharmaceutically acceptable salts preferably are pharmaceutically acceptable acid addition salts, like for example with inorganic acids, such as hydrochloric acid, sulfuric acid or a phosphoric acid, or with suitable organic carboxylic or sulfonic acids, for example aliphatic mono- or di-carboxylic acids, such as trifluoroacetic acid, acetic acid, propionic acid, glycolic acid, succinic acid, maleic acid, fumaric acid, hydroxymaleic acid, malic acid, tartaric acid, citric acid or oxalic acid, or amino acids such as arginine or lysine, aromatic carboxylic acids, such as benzoic acid, 2-phenoxy-benzoic acid, 2-acetoxy-benzoic acid, salicylic acid, 4-aminosalicylic acid, aromatic-aliphatic carboxylic acids, such as mandelic acid or cinnamic acid, heteroaromatic carboxylic acids, such as nicotinic acid or isonicotinic acid, aliphatic sulfonic acids, such as methane-, ethane- or 2-hydroxyethane-sulfonic, in particular methanesulfonic acid, or aromatic sulfonic acids, for example benzene-, p-toluene- or naphthalene-2-sulfonic acid.

Unless otherwise indicated, references to “mesilate” are used in the present invention to refer to a salt of methanesulfonic acid with a named pharmaceutical substance (such as compounds of formula (A) or (B)). Use of mesilate rather than mesylate is in compliance with the INNM (International nonproprietary names modified) issued by WHO (e.g. World Health Organization (February 2006). International Nonproprietary Names Modified. INN Working Document 05.167/3. WHO.). For example, masitinib mesilate means the methanesulfonic acid salt of masitinib.

Preferably, “masitinib mesilate” means the orally bioavailable mesilate salt of masitinib—CAS 1048007-93-7 (MsOH); C28H30N6OS.CH3SO3H; MW 594.76:

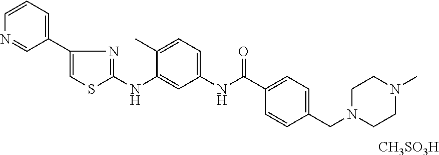

The chemical name for masitinib is 4-(4-methylpiperazin-1-ylmethyl)-N-[4-methyl-3-(4-pyridin-3ylthiazol-2-ylamino) phenyl]benzamide—CAS number 790299-79-5. Masitinib was described in U.S. Pat. No. 7,423,055 and EP1525200B1. A detailed procedure for the synthesis of masitinib mesilate is given in WO2008/098949.

Masitinib is a small molecule selectively inhibiting specific tyrosine kinases such as c-Kit, PDGFR, Lyn, Fyn and to a lesser extent the fibroblast growth factor receptor 3 (FGFR3), without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil et al., 2009, PLoS ONE 2009. 4(9):e7258). Moreover, Masitinib is a c-Kit and platelet derived growth factor receptor (PDGFR) inhibitor with a potent anti mast cell action.

Masitinib's strong inhibitory effect on wild-type and juxtamembrane-mutated c-Kit receptors, results in cell cycle arrest and apoptosis of cell lines dependent on c-Kit signaling (Dubreuil et al., 2009, PLoS ONE, 4(9):e7258). In vitro, masitinib demonstrated greater activity and selectivity against c-Kit than imatinib, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit.

In one embodiment, the method of the invention further comprises administering to the subject a therapeutically effective amount of at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof and the at least one chemotherapeutic agent are administered simultaneously, separately or sequentially.

According to one embodiment, the method of the invention thus does not consist in administering a therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof to the subject.

Indeed, the Applicant surprisingly demonstrated a synergetic effect of the combination of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate with a chemotherapeutic agent. First, the Applicant showed in in vitro tests that, despite the absence of effect of the compound alone, the administration of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate, sensitized cells to chemotherapeutic agents (see example 1). Second, the Applicant showed in in vivo data that the administration to a subject of a combination of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof such as, for example, masitinib mesilate with a chemotherapeutic agent allows increasing overall survival (see example 2).

Examples of chemotherapeutic agents that may be used in combination with the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, of the invention include, but are not limited to, adrucil (fluorouracil), Cyramza (Ramucirumab), Docetaxel, Doxorubicin Hydrochloride, Efudex (Fluorouracil), Fluoroplex (Fluorouracil), Fluorouracil, Herceptin (Trastuzumab), Mitomycin C, Mitozytrex (Mitomycin C), Mutamycin (Mitomycin C), Ramucirumab, Taxotere (Docetaxel), Trastuzumab, FU-LV combination (combination of Fluorouracil and Leucovorin Calcium), TPF combination (combination of Docetaxel (Taxotere), Cisplatin (Platinol) and Fluorouracil), vincristine, folinic acid (leucovorin), epirubicin, cisplatin, 5-fluorouracil (5-FU), etoposide, paclitaxel, irinotecan, oxaliplatin, bevacizumab and sorefanib and mixtures thereof. Examples of mixtures of chemotherapeutic agents that may be used in combination with the tyrosine kinase inhibitor of the invention include, but are not limited to, 5-FU, folinic acid and irinotecan (FOLFIRI protocol), FU-LV combination (combination of Fluorouracil and Leucovorin Calcium), TPF combination (combination of Docetaxel (Taxotere), Cisplatin (Platinol) and Fluorouracil), preferably FOLFIRI.

Preferably, the at least one chemotherapeutic agent is selected from doxorubicin, 5-fluorouracil (5-FU), irinotecan, docetaxel, trastuzumab, cisplatin, mitomycin, ramucirumab, etoposide, vincristine, folinic acid (leucovorin), and mixtures thereof.

More preferably, the at least one chemotherapeutic agent is 5-FU, irinotecan, etoposide, vincristine or is a combination of 5-FU, folinic acid and irinotecan.

In one embodiment, the at least one chemotherapeutic agent is selected from the group comprising doxorubicin, 5-fluorouracil, irinotecan, etoposide, vincristine, folinic acid, and mixtures thereof, or is a combination of 5-FU, folinic acid and irinotecan.

In one embodiment, the method of the invention comprises or consists in administering masitinib and doxorubicin to the subject. In another embodiment, the method of the invention comprises or consists in administering masitinib and 5-fluorouracil to the subject. In another embodiment, the method of the invention comprises or consists in administering masitinib and irinotecan to the subject. In another embodiment, the method of the invention comprises or consists in administering masitinib and etoposide to the subject. In another embodiment, the method of the invention comprises or consists in administering masitinib and vincristine to the subject. In another embodiment, the method of the invention comprises or consists in administering masitinib and folinic acid to the subject. In another embodiment, the method of the invention comprises or consists in administering masitinib and FOLFIRI to the subject.

In one embodiment, gastric cancer is primary gastric cancer, preferably gastric adenocarcinoma (adenocarcinoma of the stomach) or gastro-esophageal junction adenocarcinoma.

In one embodiment, the method of the invention is for treating unresectable gastric cancer. Unresectable cancers are cancers that cannot be completely removed. This might be because the tumor has grown into nearby organs or lymph nodes. Or it may have grown too close to major blood vessels, or has spread to distant parts of the body, or the patient is not healthy enough for surgery.

In one embodiment, the method of the invention is for treating metastatic gastric adenocarcinoma or metastatic gastro-esophageal junction adenocarcinoma. In another embodiment, the method of the invention is for treating unresectable gastric adenocarcinoma or unresectable gastro-esophageal junction adenocarcinoma.

In one embodiment, gastric cancer is advanced gastric cancer. In one embodiment, the term “advanced gastric cancer” corresponds to advanced gastric cancer according to the TNM (tumor, node, metastases) staging.

The TNM classification, developed and maintained by the International Union Against Cancer (UICC), is the internationally accepted standard for cancer staging. A classification is given for each component then a global stage is given (see below).

### Primary Tumor (T)

TX: Primary tumor cannot be assessed
T0: No evidence of primary tumor
Tis: Carcinoma in situ: intraepithelial tumor without invasion of the lamina propria
T1: Tumor invades lamina propria, muscularis mucosae, or submucosa
T1a: Tumor invades lamina propria or muscularis mucosae
T1b: Tumor invades submuscosa
T2: Tumor invades muscularis propria
T3: Tumor penetrates subserosal connective tissue without invasion of visceral peritoneum or adjacent structures
T4: Tumor invades serosa (visceral peritoneum) or adjacent structures
T4a: Tumor invades serosa (visceral peritoneum)
T4b: Tumor invades adjacent structures

### Regional Lymph Nodes (N)

NX: Regional nodes cannot be assessed
N0: No regional lymph node metastasis
N1: Metastasis in 1-3 regional lymph nodes
N2: Metastasis in 3-6 regional lymph nodes
N3: Metastasis in 7 regional lymph nodes
N3a: Metastasis in ≧7-15 regional lymph nodes
N3b: Metastasis in ≧16 regional lymph nodes

### Distant Metastasis (M)

M0: No distant metastasis
M1: Distant metastasis

### Staging

| Stage | T | N | M |
|---|---|---|---|
| 0 | Tis | N0 | M0 |
| IA | T1 | N0 | M0 |
| IB | T2 | N0 | M0 |
| | T1 | N1 | M0 |
| IIA | T3 | N0 | M0 |
| | T2 | N1 | M0 |
| | T1 | N2 | M0 |
| IIB | T4a | N0 | M0 |
| | T3 | N1 | M0 |
| | T2 | N2 | M0 |
| | T1 | N3 | M0 |
| IIIA | T4a | N1 | M0 |
| | T3 | N2 | M0 |
| | T2 | N3 | M0 |
| IIIB | T4b | N0 | M0 |
| | T4b | N1 | M0 |
| | T4a | N2 | M0 |
| | T3 | N3 | M0 |
| IIIC | T4b | N2 | M0 |
| | T4b | N3 | M0 |
| | T4a | N3 | M0 |
| IV | Any T | Any N | M1 |

The term “advanced gastric cancer” encompasses locally advanced gastric cancer and metastatic gastric cancer.

As used herein, the term “locally advanced gastric cancer” refers to gastric cancer that has spread locally to the area of the stomach or of the gastro-esophageal junction, such as, for example, to lymph nodes, but has not spread to distant organs and tissues. In one embodiment, locally advanced gastric cancers include stage III gastric cancers (including stages IIIA, IIIB and IIIC) according to the TNM classification.

In one embodiment, advanced gastric cancer is stage III gastric cancer, specifically stage IIIA, IIIB or IIIC gastric cancer according to the TNM classification.

In another embodiment, advanced gastric cancer is stage IV gastric cancer (i.e. metastatic gastric cancer) according to the TNM classification.

In one embodiment, the subject was not treated previously with another treatment for gastric cancer (i.e. the method of treatment of the invention is the first line treatment). In one embodiment, the subject was not treated previously with another systemic treatment for gastric cancer (wherein a systemic treatment for gastric cancer relates to a treatment with a chemotherapeutic agent). In one embodiment, the subject was not previously treated for gastric cancer by surgery or radiotherapy.

In another embodiment, the subject previously received one, two or more other treatments for gastric cancer (i.e. the method of treatment of the invention is a second line of treatment, a third line of treatment or more). In one embodiment, the subject previously received one or more other treatments for gastric cancer, but was unresponsive or did not respond adequately to these treatments, which means that there is no, or too low, therapeutic benefit induced by these treatments. Therapeutic benefits may include the fact of (1) slowing down or stopping the progression, aggravation, or deterioration of one or more symptoms of gastric cancer; (2) bringing about ameliorations of the symptoms of gastric cancer; (3) reducing the severity or incidence of gastric cancer; or (4) curing gastric cancer.

Examples of treatment for gastric cancer include, but are not limited to, gastrectomy (distal subtotal, proximal subtotal or total) with or without lymphadenectomy, tumor ablation (radiofrequency ablation (RFA), ethanol (alcohol) ablation, microwave thermotherapy, cryosurgery, endoscopic mucosal resection or nanoknife irreversible electroporation), radiation therapy, or treatment with a chemotherapeutic agent.

Examples of chemotherapeutic agents that may be used for treating gastric cancer include, but are not limited to, adrucil (fluorouracil), Cyramza (Ramucirumab), Docetaxel, Doxorubicin Hydrochloride, Efudex (Fluorouracil), Fluoroplex (Fluorouracil), Fluorouracil, Herceptin (Trastuzumab), Mitomycin C, Mitozytrex (Mitomycin C), Mutamycin (Mitomycin C), Ramucirumab, Taxotere (Docetaxel), Trastuzumab, FU-LV combination (combination of Fluorouracil and Leucovorin Calcium), TPF combination (combination of Docetaxel (Taxotere), Cisplatin (Platinol) and Fluorouracil), vincristine, folinic acid (leucovorin), epirubicin, cisplatin, 5-fluorouracil (5-FU), etoposide, paclitaxel, irinotecan, oxaliplatin, bevacizumab and sorefanib and mixtures thereof or specific combinations of chemotherapeutic agents including, without limitation, a combination of 5-FU, folinic acid and irinotecan (FOLFIRI protocol), FU-LV combination (combination of Fluorouracil and Leucovorin Calcium), TPF combination (combination of Docetaxel (Taxotere), Cisplatin (Platinol) and Fluorouracil), preferably FOLFIRI.

In one embodiment, the therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof ranges from about 1 to about 20 mg/kg/day, preferably from about 3 to about 12 mg/kg/day, and more preferably from about 4.5 to about 9 mg/kg/day. In one embodiment, the therapeutically effective amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is of about 6 mg/kg/day.

Unless otherwise indicated, any dose indicated herein refers to the amount of active ingredient as such, not to its salt form. Therefore, given that the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, dose in mg/kg/day used in the described dose regimens refers to the amount of active ingredient tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof, compositional variations of a pharmaceutically acceptable salt or solvate of tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof will not change the said dose regimens.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is orally administered.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is administered once or twice a day.

In one embodiment, the therapeutically effective amount of a chemotherapeutic agent ranges from about 1 to 5000 mg/m², preferably from about 100 to about 3000 mg/m², and more preferably from about 180 to about 2400 mg/m².

In one embodiment, the therapeutically effective amount of irinotecan ranges from about 50 to 500 mg/m², preferably from about 100 to about 400 mg/m², and more preferably from about 135 to about 350 mg/m². In one embodiment, the therapeutically effective amount of irinotecan is about 135 mg/m². In another embodiment, the therapeutically effective amount of irinotecan is about 180 mg/m². In another embodiment, the therapeutically effective amount of irinotecan is about 260 mg/m². In another embodiment, the therapeutically effective amount of irinotecan is about 350 mg/m².

In one embodiment, the therapeutically effective amount of folinic acid ranges from about 100 to about 750 mg/m², preferably from about 200 to about 500 mg/m², and more preferably from about 300 to about 400 mg/m². In one embodiment, the therapeutically effective amount of folinic acid is about 300 mg/m². In another embodiment, the therapeutically effective amount of folinic acid is about 400 mg/m².

In one embodiment, the therapeutically effective amount of 5-fluorouracil ranges from about 100 to about 4000 mg/m², preferably from about 200 to about 3000 mg/m², and more preferably from about 400 to about 2400 mg/m². In one embodiment, the therapeutically effective amount of 5-fluorouracil is of about 400 mg/m² or of about 1800 mg/m² or of about 2400 mg/m². In one embodiment, the therapeutically effective amount of 5-fluorouracil is administered in two steps, the first step at 400 mg/m² over 2 to 4 min, followed by the second step at about 1800 or about 2400 mg/m² over 46 hours.

Irinotecan is administered for example as follows: intravenous (IV) infusion, preferably at 350 or 260 mg/m² preferably at first day every 3 weeks.

FOLFIRI is administered for example as follows:
Irinotecan: continuous IV infusion, preferably at 135 or 180 mg/m² preferably over 90 minutes;
Folinic acid: IV, preferably at 300 or 400 mg/m² preferably over 120 min;
5-fluorouracil (5-FU): IV bolus, preferably at 400 mg/m² preferably over 2 to 4 min followed by continuous IV infusion, preferably at 1800 or 2400 mg/m² preferably over 46 hours.

In one embodiment, the chemotherapeutic agent is injected, such as, for example, by intravenous injection or infusion.

In one embodiment, the chemotherapeutic agent is administered once a day, or 1, 2, 3, 4, 5, 6, 7 times per week, or 1, 2, 3, 4, 5, 6, 7 times per two weeks or 1, 2, 3, 4, 5, 6, 7 times per month. In one embodiment, the administration schedule may include days or weeks periods wherein the chemotherapeutic agent is not administered. For example, the chemotherapeutic agent may be administered at first day of each week, or at first day of week 1 of a 2 or 3 weeks cycle. The skilled artisan may easily adapt the administration schedule, according, for example, to previous treatment history of the patient, to the severity of the disease to be treated, to the nature of the chemotherapeutic agent and the like.

Another object of the invention is a composition comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate.

In one embodiment, the composition of the invention further comprises at least one chemotherapeutic agent. In one embodiment, the at least one chemotherapeutic agent is selected from doxorubicin, 5-fluorouracil, irinotecan, folinic acid (leucovorin), etoposide, vincristine, and mixtures thereof.

In one embodiment, the composition of the invention comprises or consists in masitinib and doxorubicin. In another embodiment, the composition of the invention comprises or consists in masitinib and 5-fluorouracil. In another embodiment, the composition of the invention comprises or consists in masitinib and irinotecan. In another embodiment, the composition of the invention comprises or consists in masitinib and folinic acid. In another embodiment, the composition of the invention comprises or consists in masitinib and etoposide. In another embodiment, the composition of the invention comprises or consists in masitinib and vincristine. In another embodiment, the composition of the invention comprises or consists in masitinib and FOLFIRI.

Another object of the invention is a pharmaceutical composition comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof in combination with at least one pharmaceutically acceptable carrier.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate.

In one embodiment, the pharmaceutical composition of the invention further comprises at least one chemotherapeutic agent.

In one embodiment, the at least one chemotherapeutic agent is selected from doxorubicin, 5-fluorouracil, irinotecan, folinic acid, etoposide, vincristine, and mixtures thereof.

In one embodiment, the pharmaceutical composition of the invention comprises or consists in masitinib and doxorubicin in combination with at least one pharmaceutically acceptable carrier. In another embodiment, the pharmaceutical composition of the invention comprises or consists in masitinib and 5-fluorouracil in combination with at least one pharmaceutically acceptable carrier. In another embodiment, the pharmaceutical composition of the invention comprises or consists in masitinib and irinotecan in combination with at least one pharmaceutically acceptable carrier. In another embodiment, the pharmaceutical composition of the invention comprises or consists in masitinib and folinic acid in combination with at least one pharmaceutically acceptable carrier. In another embodiment, the pharmaceutical composition of the invention comprises or consists in masitinib and etoposide in combination with at least one pharmaceutically acceptable carrier. In another embodiment, the pharmaceutical composition of the invention comprises or consists in masitinib and vincristine in combination with at least one pharmaceutically acceptable carrier. In another embodiment, the pharmaceutical composition of the invention comprises or consists in masitinib and FOLFIRI in combination with at least one pharmaceutically acceptable carrier.

Another object of the invention is a medicament comprising a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof.

In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate.

In one embodiment, the medicament of the invention further comprises at least one chemotherapeutic agent. In one embodiment, the chemotherapeutic agent is selected from doxorubicin, 5-fluorouracil, irinotecan, folinic acid, etoposide, vincristine, and mixtures thereof.

In one embodiment, the medicament of the invention comprises or consists in masitinib and doxorubicin. In another embodiment, the medicament of the invention comprises or consists in masitinib and 5-fluorouracil. In another embodiment, the medicament of the invention comprises or consists in masitinib and irinotecan. In another embodiment, the medicament of the invention comprises or consists in masitinib and folinic acid. In another embodiment, the medicament of the invention comprises or consists in masitinib and etoposide. In another embodiment, the medicament of the invention comprises or consists in masitinib and vincristine. In another embodiment, the medicament of the invention comprises or consists in masitinib and FOLFIRI.

Another object of the invention is a kit of part comprising two parts, wherein the first part comprises a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof and wherein the second part comprises at least one chemotherapeutic agent. In one embodiment, the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof is masitinib, preferably masitinib mesilate. In one embodiment, the at least one chemotherapeutic agent is selected from doxorubicin, 5-fluorouracil, irinotecan, folinic acid, etoposide, vincristine, and mixtures thereof.

In one embodiment, the first part of the kit of part of the invention comprises masitinib and the second part comprises doxorubicin. In another embodiment, the first part of the kit of part of the invention comprises masitinib and the second part comprises 5-fluorouracil. In another embodiment, the first part of the kit of part of the invention comprises masitinib and the second part comprises irinotecan. In another embodiment, the first part of the kit of part of the invention comprises masitinib and the second part comprises folinic acid. In another embodiment, the first part of the kit of part of the invention comprises masitinib and the second part comprises etoposide. In another embodiment, the first part of the kit of part of the invention comprises masitinib and the second part comprises vincristine. In another embodiment, the first part of the kit of part of the invention comprises masitinib and the second part comprises FOLFIRI.

In one embodiment of the invention, the composition, pharmaceutical composition, medicament or kit of part of the invention comprises an amount of a tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof ranging from about 10 to about 500 mg, preferably from about 50 to about 300 mg, and more preferably from about 100 to about 200 mg.

In one embodiment of the invention, the composition, pharmaceutical composition, medicament or kit of part of the invention comprises an amount of masitinib ranging from about 10 to about 500 mg, preferably from about 50 to about 300 mg, and more preferably from about 100 to about 200 mg. In one embodiment, the composition, pharmaceutical composition, medicament or kit of part of the invention comprises an amount of masitinib of about 100 mg (corresponding to an amount of masitinib mesilate of about 119.3 mg). In another embodiment, the composition, pharmaceutical composition, medicament or kit of part of the invention comprises an amount of masitinib of about 200 mg (corresponding to an amount of masitinib mesilate of about 238.5 mg).

In one embodiment, the composition, pharmaceutical composition, medicament of the invention or the first and/or second part of the kit of part of the invention is in a form adapted for oral administration.

Examples of forms adapted for oral administration include, but are not limited to, tablets, orodispersing/orodispersing tablets, effervescent tablets, powders, granules, pills (including sugarcoated pills), dragees, capsules (including soft gelatin capsules), syrups, liquids, gels or other drinkable solutions, suspensions, slurries, liposomal forms and the like.

In one embodiment, the composition, pharmaceutical composition, medicament of the invention or the first and/or second part of the kit of part of the invention is in a form adapted for injection, such as, for example, for intramuscular, subcutaneous, intradermal, transdermal or intravenous injection or infusion.

Examples of forms adapted for injection include, but are not limited to, solutions, such as, for example, sterile aqueous solutions, dispersions, emulsions, suspensions, solid forms suitable for using to prepare solutions or suspensions upon the addition of a liquid prior to use, such as, for example, powder, liposomal forms and the like.

In one embodiment, the part of the kit of part comprising the tyrosine kinase inhibitor or mast cell inhibitor that is an inhibitor of kinase activity selected from the tyrosine kinases of c-Kit, platelet-derived growth factor receptor (PDGFR), LYN, FYN or any combination thereof or a pharmaceutically acceptable salt or solvate thereof is in a form adapted for oral administration, while the second part of the kit of part (comprising the chemotherapeutic agent) is in a form adapted for injection.

The present invention further relates to a composition, a pharmaceutical composition, a medicament or a kit of part as described hereinabove for treating gastric cancer, or for use in treating gastric cancer.

In one embodiment of the invention, the composition, pharmaceutical composition, medicament or kit of part as described hereinabove is for use in the method for treating gastric cancer of the invention.

Masitinib is a small molecule drug, selectively inhibiting specific tyrosine kinases such as c-Kit, platelet-derived growth factor receptor (PDGFR), LYN, and FYN, without inhibiting, at therapeutic doses, kinases associated with known toxicities (i.e. those tyrosine kinases or tyrosine kinase receptors attributed to possible tyrosine kinase inhibitor cardiac toxicity, including ABL, KDR and Src) (Dubreuil, 2009).

In one embodiment, the method of the invention comprises inhibiting tyrosine kinases, preferably selected from the group consisting of c-Kit, LYN, FYN and PDGFR α and β, thereby treating gastric cancer.

The present invention thus also relates to a method for inhibiting tyrosine kinases, preferably selected from the group consisting of c-Kit, LYN, FYN and PDGFR α and β in a gastric cancer patient, thereby treating gastric cancer, wherein said method comprises administering a therapeutically effective amount of masitinib or a pharmaceutically acceptable salt or solvate thereof.

In one embodiment, the method of the invention comprises inhibiting c-Kit. In one embodiment, the method of the invention comprises inhibiting LYN. In one embodiment, the method of the invention comprises inhibiting FYN. In one embodiment, the method of the invention comprises inhibiting PDGFR α and β, in particular inhibiting the in vitro protein kinase activity of PDGFR-α and β.

Masitinib's main kinase target is c-Kit, for which it has been shown to exert a strong inhibitory effect on wild-type and juxtamembrane-mutated c-Kit receptors, resulting in cell cycle arrest and apoptosis of cell lines dependent on c-Kit signaling (Dubreuil et al., 2009, PLoS ONE, 4(9):e7258). In vitro, masitinib demonstrated high activity and selectivity against c-Kit, inhibiting recombinant human wild-type c-Kit with an half inhibitory concentration (IC₅₀) of 200±40 nM and blocking stem cell factor-induced proliferation and c-Kit tyrosine phosphorylation with an IC₅₀ of 150±80 nM in Ba/F3 cells expressing human or mouse wild-type c-Kit. In addition to its anti-proliferative properties, masitinib can also regulate the activation of mast cells through its targeting of Lyn and Fyn, key components of the transduction pathway leading to IgE induced degranulation (Gilfillan et al., 2006, Nat Rev Immunol, 6:218-230) (Gilfillan et al., 2009, Immunological Reviews, 228:149-169). This can be observed in the inhibition of FccRI-mediated degranulation of human cord blood mast cells (Dubreuil et al., 2009, PLoS ONE; 4(9):e7258). Masitinib is also an inhibitor of PDGFR α and β receptors. Recombinant assays show that masitinib inhibits the in vitro protein kinase activity of PDGFR-α and β with IC₅₀ values of 540±60 nM and 800±120 nM. In Ba/F3 cells expressing PDGFR-α, masitinib inhibited PDGF-BB-stimulated proliferation and PDGFR-α tyrosine phosphorylation with an IC₅₀ of 300±5 nM.

In oncology indications for which masitinib's tyrosine kinase targets are not the main oncogenic drivers the main mode of action of masitinib is through modulation of the immune response. Experimental data indicate that masitinib is capable of modulating the immune response in such a way as to positively impact on physiological disturbances such as oxidative stress (Adenis A, et al. Ann Oncol. 2014 Sep.; 25(9):1762-9). In particular, masitinib induces an anti-tumoral Th1 immune response via recruitment of macrophages with a potential antitumoral activity within the tumor and also modulates the tumor microenvironment through its inhibition of mast cell activity with reduced release of M2-polarizing cytokines (protumoral), as well as other factors favoring metastasis and angiogenesis. Subsequent antitumoral activity within the tumor and tumor microenvironment confers conditions conducive to retarding aggressiveness and dissemination of the tumor in a manner independent of association with any particular active chemotherapeutic agent.

More specifically, recent experimental data demonstrate that masitinib induces an anti-tumoral Th1 immune response, due to the following mechanisms of action: (i) masitinib acts on macrophages, by increasing both the release of chemoattractants which attracts macrophages to the tumor site (such as, for example, CCL2), and the expression of M1-polarizing cytokines, such as, for example, CXCL9 and CXCL10; (ii) masitinib inhibits mast cell proliferation and degranulation and thereby reduces the release of M2-polarizing cytokines, as well as other factors favoring metastasis and angiogenesis (such as VEGF); and (iii) masitinib increases cytotoxic NK activity and IFN gamma release through its interaction with dendritic cells.

In one embodiment, the method of the invention comprises inducing an anti-tumoral Th1 immune response, thereby treating gastric cancer.

The present invention thus also relates to a method for inducing an anti-tumoral Th1 immune response in a gastric cancer patient, thereby treating gastric cancer, wherein said method comprises administering a therapeutically effective amount of masitinib or a pharmaceutically acceptable salt or solvate thereof.

In one embodiment, the method of the invention comprises increasing the release of chemoattractants which attracts macrophages to the tumor site (such as, for example, CCL2), and/or increasing the expression of M1-polarizing cytokines, such as, for example, CXCL9 and CXCL10.

In one embodiment, the method of the invention comprises inhibiting mast cell proliferation and degranulation and thereby reducing the release of M2-polarizing cytokines, as well as other factors favoring metastasis and angiogenesis (such as VEGF).

In one embodiment, the method of the invention comprises increasing cytotoxic NK activity and IFN gamma release through the interaction of masitinib with dendritic cells.

In one embodiment, the method of the invention comprises (i) inhibiting tyrosine kinases, preferably selected from the group consisting of c-Kit, LYN, FYN and PDGFR α and β and (ii) inducing an anti-tumoral Th1 immune response, thereby treating gastric cancer.

The present invention thus also relates to a method for (i) inhibiting tyrosine kinases, preferably selected from the group consisting of c-Kit, LYN, FYN and PDGFR α and β and (ii) inducing an anti-tumoral Th1 immune response, in a gastric cancer patient, thereby treating gastric cancer, wherein said method comprises administering a therapeutically effective amount of masitinib or a pharmaceutically acceptable salt or solvate thereof.

In one embodiment, the method of the invention comprises inhibiting c-Kit signaling pathways. In one embodiment, the method of the invention comprises inhibiting LYN signaling pathways. In one embodiment, the method of the invention comprises inhibiting FYN signaling pathways. In one embodiment, the method of the invention comprises inhibiting PDGF signaling pathways. As used herein, the terms “signaling pathway” refers to a group of molecules in a cell that work together to control one or more cell functions (such as, for example cell division or cell death). After the first molecule in a pathway receives a signal, it activates another molecule. This process is repeated until the last molecule is activated and the cell function is carried out.

### EXAMPLES

The present invention is further illustrated by the following examples.

### Example 1

### Masitinib Sensitizes Gastric Cancer Cell Lines to Chemotherapies

### Materials and Methods

### Compounds

Masitinib (having the molecular formula C₂₈H₃₀N₆OS.CH₄O₃S) presents as a white powder. Stock solution of 20 mM in DMSO was stored at −80° C. The other agents were purchased from Sigma Aldrich Corporation and are poisons of microtubules (Vincristin), anti-topoisomerase I (Irinotecan), anti-topoisomerase II (Etoposide), and alkylant agents such as platinum salt (Carboplatin). These agents are commonly used as treatment for various tumor types either as single agent or in combination regimen.

### Cell Culture

Gastric cancer cell lines AGS and HGC-27 (purchased from Cell Line Service, Germany) were cultured as monolayers in DMEM Glutamax and DMEM:F12 (1/1 mixture) Glutamax respectively, supplemented with 100 U/mL penicillin and 100 μg/mL streptomycin, and 10% v/v heat-inactivated fetal calf serum (Eurobio ref CVFSVF00-01 Lot S35531-1135) under standard culture conditions (5% CO2, 95% air in humidified chamber at 37° C.).

During proliferation assay, all cells were grown in medium containing 1% FCS.

### Experimental Design

Colorimetric cell proliferation and viability assay (reagent CellTiter-Blue purchased from Promega cat N°G8081)—Cells were washed once and resuspended in DMEM/DMEM:F12 1% FCS. Cells were plated at 1.10⁴/50 μl per well of a 96 well plate. Drug dilutions were prepared in a 96 well plate and obtained by sequential dilutions of masitinib in DMEM/DMEM:F12 1% FCS. Treatment was started by the addition of 50 μl of a 2× concentrated drug solution to a final volume of 100 μl. For treatment with combination of masitinib and cytotoxic agents, the cells were first resuspended in medium DMEM/DMEM:F12 1% FCS containing masitinib at the concentrations of 0, 2, 5 and 10 μM, plated as before in 96 wells plates and placed in the incubator overnight (o/n) before treatment with cytotoxic agents. Cytotoxic agent treatment was initiated by addition of 50 μl of a 2× drug dilution (and containing the respective masitinib drug concentration) to a final volume of 100 μl. Masitinib final concentrations remained 0, 2, 5 and 10 μM. After incubating for 72 hours at 37° C., 10 μl of a 1/2 dilution of CellTiter-Blue reagent was added to each well and the plates were returned to the incubator for an additional 4 hours. The fluorescence intensity from the CellTiter-Blue reagent is proportional to the number of viable cells and data were recorded (544Ex/590Em) using a POLARstar OMEGA microplate reader (BMG Labteck Sarl). A background control without cells was used as a blank. The positive control of the assay corresponds to the cell proliferation obtained in the absence of drug treatment (100% proliferation). Each sample was done in duplicate, the absorbance values were transferred to an excel file, the average and standard deviation of the duplicates were calculated and expressed as a percentage of the proliferation obtained in absence of treatment. The results presented in this study are representative of a minimum of 3 experiments. The sensitization factor/Index is calculated by dividing the IC₅₀ of the chemotherapeutic agent alone by the IC₅₀ of the chemotherapeutic agent used in combination with masitinib.

### Results

In order to assess the benefits of using masitinib in combination therapy for cancer treatment, we performed preclinical studies involving tumour cell lines. The project consisted in testing the ability of masitinib to sensitize gastric cancer cell lines AGS and HGC-27 to cytotoxic agents using in vitro proliferation assays.

We used a large panel of cytotoxic agents that exert their cytotoxicity through different mechanisms. These agents included the conventional chemotherapeutic agent fluorouracil (5-FU) as well as non-standard chemotherapeutic agents such as irinotecan, etoposide, and vincristin.

### Masitinib is not Active as Single Agent

Gastric cancer cell lines AGS and HGC-27 were first analyzed for their sensitivity to masitinib when used as single agent. This analysis showed that gastric cancer cell lines were not sensitive to masitinib (IC₅₀=5-10 μM) suggesting that proliferation/survival of the cell lines examined may not be dependent on the expression of masitinib main targets PDGFRβ and c-Kit. Based on these data, masitinib was used at concentrations of 5 and 10 μM in the following combinatory experiments.

### Masitinib Sensitizes Gastric Cancer Cells to 5-FU

To determine the IC₅₀ concentration of 5-FU used in association with masitinib, gastric cancer cell lines grown in 1% FCS were pre-treated with masitinib (at 2, 5 or 10 μM) for about 12-16 hours before being exposed to different doses of the chemotherapeutic agent.

Results are shown in Table 1.

TABLE 1
Masitinib sensitizes gastric cell lines to 5-FU

| IC₅₀ μM | | | |
|---|---|---|---|
| Cell lines | 5-FU | 5-FU plus masitinib | SI |
| AGS | 100 | 5 | 20 |
| HCG-27 | 1000 | 20-100 | 10-50 |

SI = Sensitization Index

The cell lines AGS and HGC-27 are respectively sensitive and resistant to 5-FU (reported Cmax=60-200 μM) and the addition of masitinib significantly enhances the sensitivity of both cell lines to the agent.

### Masitinib Sensitizes Gastric Cancer Cells to Irinotecan

The ability of masitinib to sensitize gastric cancer cell lines to the action of anti-topoisomerase I agent irinotecan was next assessed. Summary of the results is presented in Table 2.

TABLE 2
Masitinib sensitizes gastric cancer cell lines to irinotecan

| IC₅₀ (μM) | | | |
|---|---|---|---|
| Cell lines | Irinotecan | Irinotecan plus masitinib | SI |
| AGS | 20 | 0.5 | 40 |
| HCG-27 | >100 | 5-50 | 2-20 |

SI = Sensitization Index

Interestingly both cell lines appear to be resistant to irinotecan and a good sensitization is observed when masitinib was added. The presence of masitinib lowers the IC₅₀ of irinotecan to clinically achievable concentrations (approximate Cmax measured in plasma of 1-10 μM).

### Masitinib Sensitizes Gastric Cancer Cell Lines to Etoposide

We next tested the ability of masitinib to sensitize gastric cancer cell lines to the action of anti-topoisomerase II agent etoposide. Results are shown in Table 3.

TABLE 3
Masitinib sensitizes gastric cancer cell lines to etoposide

| IC₅₀ (μM) | | | |
|---|---|---|---|
| Cell lines | Etoposide | Etoposide plus masitinib | SI |
| AGS | 10 | 1 | 10 |
| HCG-27 | 20 | 1-20 | 1-20 |

SI = Sensitization Index

Interestingly both cell lines were sensitized to etoposide when masitinib was added. The presence of masitinib lowers the IC₅₀ of etoposide to clinically achievable concentrations (approximate Cmax measured in plasma of 34 μM).

### Masitinib Sensitizes Gastric Cancer Cells to Vincristin

We next assessed the ability of masitinib to sensitize gastric cancer cell lines to the action of the alkaloid agent vincristin. Results are shown in Table 4.

TABLE 4
Masitinib sensitizes gastric cancer cell lines to vincristin

| IC₅₀ (μM) | | | |
|---|---|---|---|
| Cell lines | Vincristin | Vincristin plus masitinib | SI |
| AGS | >1 | 0.05 | 20 |
| HCG-27 | 0.1-1 | 0.1-0.01 | 10 |

SI = Sensitization Index

Although the cell lines exhibit resistance to vincristin the addition of masitinib significantly potentiates the action of the chemotherapeutic agent.

These results thus demonstrate that, surprisingly, masitinib is able to sensitize gastric cancer cell lines to cytotoxic agents in vitro, despite its absence of activity when used alone. Therefore, these results highlight the synergistic effect of the combination of masitinib and cytotoxic agents.

### Example 2

### Treatment of Gastric Adenocarcinoma with a Combination of Masitinib and Irinotecan (Phase 1/2 Study)

A prospective, multicenter, open-label, randomised, uncontrolled, phase 1/2 study has been conducted to evaluate efficacy and safety of masitinib in association with irinotecan after a first-line of cytotoxic chemotherapy of patients suffering from advanced-stage gastric or gastro-esophageal junction adenocarcinoma.

### Methodology

Fourteen patients resistant to at least one first line of chemotherapy (radiotherapy, chemotherapy, chemoradiotherapy or targeted therapy) have been enrolled. In this open-label study, masitinib was administered orally at the daily dose of 6 mg/kg in two intakes, in combination with irinotecan given at the dose of 260 or 350 mg/m² every three weeks.

### Results

Overall survival (OS) is defined as the time from first treatment intake to the date of documented death. If death was not observed, data on OS were censored at the last date patient was known to be alive. Median OS was analyzed using Kaplan-Meier and was given with its confidence interval (CI) of 95%.

In this first phase 1/2 study, last available analysis shows overall survival with masitinib in combination with irinotecan is 15.1 months (95% CI[4.2; 15.6]) while the benchmark for a second-line of chemotherapy (L2) is around 7 months (Roy A C, et al. Ann Oncol. 2013 Jun.; 24(6):1567-73) (Rosati G, et al. World J Gastroenterol. 2009 Jun. 14; 15(22): 2689-92).

In conclusion, patients with advanced-stage gastric or gastro-esophageal junction adenocarcinoma resistant to first-line chemotherapy and treated with the combination of masitinib plus irinotecan showed an increased OS compared to the published data.

A phase 3 study has been initiated to evaluate efficacy and safety of masitinib in combination with irinotecan versus placebo in combination with irinotecan in patients with advanced-stage gastric or esophageal adenocarcinoma and who relapsed after a first line chemotherapy.

### Example 3

### Phase 2 Study of Masitinib in Combination with Irinotecan, 5-Fluorouracil and Folinic Acid (FOLFIRI Protocol)

A prospective, multicenter, open-label, randomised, uncontrolled, phase 1/2 study has been conducted to evaluate efficacy and safety of masitinib in association with irinotecan, 5-fluorouracil (5-FU) and folinic acid (FOLFIRI protocol) as second-line of cytotoxic chemotherapy in patients with advanced-stage gastric or gastro-esophageal junction adenocarcinoma.

### Methodology

Thirteen patients resistant to at least one first line of chemotherapy (radiotherapy, chemotherapy, chemoradiotherapy or targeted therapy) have been enrolled. In this open-label study, masitinib at 6 mg/kg/day was administered in association with FOLFIRI protocol i.e., every 2 weeks:
Irinotecan, continuous infusion at 180 mg/m² or 135 mg/m² over 90 min;
Folinic acid, IV at 400 mg/m² or 300 mg/m² over 120 min;
5-fluorouracil IV bolus at 400 mg/m² over 2 to 4 min, followed by continuous IV infusion at 2400 mg/m² or 1800 mg/m² over 46 hours.

### Results

In this phase 1/2 study, last available analysis shows overall survival with masitinib in combination with FOLFIRI is 10.8 months (95% CI[5.0; 22.5]) while the benchmark for a second-line of chemotherapy (L2) is around 7 months (Roy A C, et al. Ann Oncol. 2013 Jun.; 24(6):1567-73) (Rosati G, et al. World J Gastroenterol. 2009 Jun. 14; 15(22): 2689-92).

In conclusion, patients with advanced-stage gastric or gastro-esophageal junction adenocarcinoma resistant to a first-line chemotherapy and treated with the combination of masitinib plus irinotecan, 5-FU and folinic acid showed an increased OS compared to the published data.

1. A method for treating gastric cancer in a subject in need thereof, comprising administering to the subject a therapeutically effective amount of a tyrosine kinase inhibitor or mast cell inhibitor or a pharmaceutically acceptable salt or solvate thereof, optionally in combination with a therapeutically effective amount of at least one chemotherapeutic agent.

2. The method according to claim 1, wherein the tyrosine kinase inhibitor or mast cell inhibitor is an inhibitor of c-Kit, Lyn, Fyn and/or PDGFR α and β.

3. The method according to claim 1, wherein the tyrosine kinase inhibitor or mast cell inhibitor is masitinib or a pharmaceutically acceptable salt or solvate thereof.

4. The method according to claim 1, wherein the tyrosine kinase inhibitor or mast cell inhibitor is masitinib mesilate.

5. The method according to claim 1, for improving survival and/or life expectancy of the subject.

6. The method according to claim 1, comprising sensitizing to a chemotherapeutic agent or restoring sensitivity to a chemotherapy in the subject.

7. The method according to claim 1, wherein gastric cancer is locally advanced gastric cancer or metastatic gastric cancer.

8. The method according to claim 1, wherein gastric cancer is primary gastric cancer, gastric adenocarcinoma or gastro-esophageal junction adenocarcinoma.

9. The method according to claim 1, wherein gastric cancer is unresectable gastric adenocarcinoma or unresectable gastro-esophageal junction adenocarcinoma.

10. The method according to claim 1, wherein gastric cancer is relapsed or is refractory gastric cancer.

11. The method according to claim 1, wherein the subject is naïve to anti-gastric cancer treatments, or wherein gastric cancer relapsed after at least one anti-gastric cancer treatment, or after two or more anti-gastric cancer treatments.

12. The method according to claim 11, wherein anti-gastric cancer treatment include treatment with one or more chemotherapeutic agents, selected from adrucil (fluorouracil), Cyramza (Ramucirumab), Docetaxel, Doxorubicin Hydrochloride, Efudex (Fluorouracil), Fluoroplex (Fluorouracil), Fluorouracil, Herceptin (Trastuzumab), Mitomycin C, Mitozytrex (Mitomycin C), Mutamycin (Mitomycin C), Ramucirumab, Taxotere (Docetaxel), Trastuzumab, FU-LV combination (combination of Fluorouracil and Leucovorin Calcium), TPF combination (combination of Docetaxel (Taxotere), Cisplatin (Platinol) and Fluorouracil), vincristine, folinic acid (leucovorin), epirubicin, cisplatin, 5-fluorouracil (5-FU), etoposide, paclitaxel, irinotecan, oxaliplatin, bevacizumab and sorefanib and mixtures thereof, or specific combinations of chemotherapeutics including the combination of 5-FU, folinic acid and irinotecan (FOLFIRI protocol), FU-LV combination (combination of Fluorouracil and Leucovorin Calcium), TPF combination (combination of Docetaxel (Taxotere), Cisplatin (Platinol) and Fluorouracil).

13. The method according to claim 1, wherein the at least one chemotherapeutic agent is selected from 5-fluorouracil, irinotecan, folinic acid, etoposide, vincristine and mixtures thereof, such as, for example, the combination of 5-fluorouracil, irinotecan, folinic acid (FOLFIRI protocol).

14. The method according to claim 1, wherein the therapeutically effective amount of the tyrosine kinase inhibitor is about 6 mg/kg/day.

15. The method according to claim 1, wherein the tyrosine kinase inhibitor is orally administered.

16. The method according to claim 1, wherein the tyrosine kinase inhibitor is administered twice daily.

17. A method for inhibiting tyrosine kinases, selected from the group consisting of c-Kit, LYN, FYN and PDGFR α and β and for inducing an anti-tumoral Th1 immune response, in a gastric cancer patient, thereby treating gastric cancer, wherein said method comprises administering a therapeutically effective amount of a tyrosine kinase inhibitor or mast cell inhibitor, preferably masitinib, or a pharmaceutically acceptable salt or solvate thereof, optionally in combination with a therapeutically effective amount of a chemotherapeutic agent.

18. A pharmaceutical composition comprising a tyrosine kinase inhibitor or mast cell inhibitor or a pharmaceutically acceptable salt or solvate thereof and a chemotherapeutic agent, in combination with at least one pharmaceutically acceptable carrier, wherein said tyrosine kinase inhibitor or mast cell inhibitor is preferably masitinib mesilate, and wherein said chemotherapeutic agent is preferably selected from doxorubicin, 5-fluorouracil, irinotecan, folinic acid, etoposide, vincristine and mixtures thereof.

19. A medicament comprising a tyrosine kinase inhibitor or mast cell inhibitor or a pharmaceutically acceptable salt or solvate thereof and a chemotherapeutic agent, wherein said tyrosine kinase inhibitor or mast cell inhibitor is preferably masitinib mesilate, and wherein said chemotherapeutic agent is preferably selected from doxorubicin, 5-fluorouracil, irinotecan, folinic acid, etoposide, vincristine and mixtures thereof.

20. A kit of part comprising, in a first part, a tyrosine kinase inhibitor or mast cell inhibitor or a pharmaceutically acceptable salt or solvate thereof, preferably wherein said tyrosine kinase inhibitor or mast cell inhibitor is masitinib mesilate, and, in a second part, a chemotherapeutic agent, preferably selected from doxorubicin, 5-fluorouracil, irinotecan, folinic acid, etoposide, vincristine and mixtures thereof.
